(19) United States
(12) Patent Application Publication
(10) Pub. No.: US 2003/0095993 A1
(43) Pub. Date: May 22, 2003

(54) Gel-infused sponges for tissue repair and augmentation

(76) Inventors: Hanne Bentz, Newark, CA (US); A. Minerva Garcia, Chula Vista, CA (US); Jeffrey A. Hubbell, Zumikon (CH)

Correspondence Address: FISH & NEAVE, 1251 AVENUE OF THE AMERICAS, 50TH FLOOR, NEW YORK, NY, 10020-1105, US

(21) Appl. No.: 10/207,439

(22) Filed: Jul. 26, 2002

Related U.S. Application Data
(63) Continuation of application No. PCT/US01/02837, filed on Jan. 26, 2001.
(60) Provisional application No. 60/178,646, filed on Jan. 28, 2000.

(51) Int. Cl. A61F 2/00

(52) U.S. Cl. 424/426

(57) ABSTRACT

Gel-infused sponge matrix comprising an absorbable sponge material, a gel and an active ingredient are disclosed, as are methods of enhancing tissue repair, regeneration or augmentation using the gel-infused sponge.

### BACKGROUND OF THE INVENTION

Collagen Materials

Collagen is known in the art and has been used in different forms for many purposes including the promotion of cell growth and the delivery of pharmaceuticals.

Collagen has been used by itself and in combination with other agents to promote wound healing, tissue growth and delivery of pharmaceuticals. Agents for wound healing include bioactive agents, plasticizers, stabilizers, biopolymer, and pharmaceutical combinations. Examples of agents used in conjunction with collagen are fibrinogen and thrombin. (Steffan et al. European Patent Application 069260, published Jan. 12, 1983; Zimmerman et al. U.S. Pat. No. 4,453,939, issued Jun. 12, 1984; Leibovich et al. U.S. Pat. No. 4,808,402, issued February 1989; Yannas and Burke, J. Biomed. Mat. Res. 14:68-81 (1980); Pachence et al., Med. Device and Diag. Ind., 9:49-55 (1987); Song et al. U.S. Pat. No. 5,512,301, issued Apr. 30, 1996; Rosenthal et al. U.S. Pat. No. 5,565,210 issued Oct. 15, 1996; Rosenthal et al. U.S. Pat. No. 5,466,462, issued Nov. 14, 1995; Silver et al. U.S. Pat. No. 4,703,108, issued Oct. 27, 1987).

Crosslinked Gels

Cross-linked gels have been used in different forms for many purposes, including the delivery of cells and bioactive agents. An example of this is the polymerization of water soluble macromers containing free radical polymerizable groups such as carbon-carbon double and triple bonds (Hubbell et al., U.S. Pat. No. 5,843,743). These water soluble macromers may form gels by UV or visible light irradiation (Hubbell et al., U.S. Pat. No. 5,801,033). This includes gels which consist of both a core and extensions, where the extensions are designed to reduce tissue, cell and protein adhesions with the gel (Hubbell et al., U.S. Pat. No. 5,626,863). Cells may also be delivered in a water soluble polyethylene oxide gel (Hubbell et al., U.S. Pat. No. 5,380,536).

A process for forming an oriented structure within a biocompatible, bioabsorbable gel has been used in the art (Barrows et al., U.S. Pat. No. 5,856,367). A method for forming a gel from serum albumin which reacts with a bifunctional water-soluble cross-linking agent is described in Barrows et al., U.S. Pat. No. 5,583,114.

Methods for making a gel from collagen and a bifunctional polyethylene glycol have been used in the art (Rhee et al., U.S. Pat. No. 5,550,187, U.S. Pat. No. 5,523,348, U.S. Pat. No. 5,328,955, U.S. Pat. No. 5,304,595). A method for forming a cross-linked gel using chemically modified glycosaminoglycans, and a bifunctional, water-soluble cross-linking agent has also been used in the art (Rhee et al., U.S. Pat. No. 5,510,418). These gels may also contain collagen.

Bioactive Agents

A bioactive agent is any compound, chemical, biological or pharmaceutical, that has an effect on cells and therefore a biological effect. An example of a pharmaceutical agent is suramin which inhibits vascular ingrowth. Other bioactive agents are materials such as growth factors for the promotion of cell recruitment, growth and transformation. Agents such as these have been used in the art (see e.g., Rizzino, A., Dev. Biol., 130, pp. 411-22 (1988)). Such growth factors include, e.g., BMP's, TGF-β's, EGF and FGFs. See, e.g., Seyedin et al., Proc. Natl. Acad. Sci. U.S.A., 82, pp. 2267-71 (1985); Seyedin et al. J. Biol. Chem., 261, pp. 5693-95 (1986); Seyedin et al. J. Biol. Chem., 262, pp. 1946-49 (1987); Ginineq-Gallego et al., Biochem. Biophys. Res. Commun., 135, pp. 541-48 (1986); Thomas et al., Trends Biochem. Sci., 11, pp. 81-84 (1986)).

Transforming Growth Factor Beta (TGF-β) is a member of the family of TGF-β polypeptides (Derynck, R. et al., Nature, 316, pp. 701-705 (1985); Roberts et al., “The transforming growth factors—β's”, In Peptide growth factors and their receptors I (Berlin: Springer Verlag, 1990), p. 419)) or derivatives thereof, obtained from natural, synthetic or recombinant sources, which exhibits the characteristic TGF-β ability to stimulate normal rat kidney (NRK) cells to grow and form colonies in a soft agar assay (Roberts et al., “Purification of Type β Transforming Growth Factors From Nonneoplastic Tissues”, in Methods for Preparation of Media, Supplements, and Substrata for Serum-Free Animal Cell Culture (New York: Alan R. Liss, Inc., 1984)) and which is capable of inducing transformation of mesenchymal or fibroblast like cells into chondrocytes as evidenced by the ability to induce or stimulate production of cartilage-specific proteoglycans and type II collagen by cells in vitro (Seyedin et al., 1985, supra).

Fibroblast Growth Factors (FGFs) may be classified as acidic (aFGF) or basic (bFGF) depending on their isoelectric points. FGFs are a family of polypeptides (Ginineq-Gallego et al., Biochem. Biophys. Res. Commun., 135, pp. 541-48 (1986); Thomas et al., Trends Biochem. Sci., 11, pp. 81-84 (1986)) or derivatives thereof, obtained from natural, synthetic or recombinant sources, which exhibit the ability to stimulate DNA synthesis and cell division in vitro in a variety of cells, including primary fibroblasts, chondrocytes, vascular and coreal, and glial cells (Thomas et al., 1986, supra; for assays see, e.g., Giminez-Gallego et al., 1986 supra; Canalis et al., J. Clin. Invest., 81, pp. 1572-77 (1988)).

Other growth factors that initiate or stimulate formation of bone or cartilage have also been identified. Such growth factors are proteins that belong to the transforming growth factor beta (TGF-β) family of growth and differentiation factors (Wozney et al., Clinical Orthopaedics and Rel. Res., 346, 26-37 (1998); Schmitt, J., Orthopaedic Res., 17, 269-278 (1999); Reddi, Curr. Opin. Genet. Dev., 4, 737-744 (1994)). Examples are transforming growth factor beta 1, 2 and 3 (TGF-β1, 2 and 3), bone morphogenetic proteins (all BMP's except BMP-1) and growth and differentiation factors, e.g.: GDF-5, 6 and 7.

Repair of Articular Cartilage

Two primary approaches have been used for the biological repair of articular cartilage. The first approach uses autologous chondrocytes transplanted into the lesion to induce repair. (Grande et al., J. Orthop. Res. 7, 208-214 (1989); Brittberg et al., New Engl. J. Med. 331, 889-895 (1994); Shortkroff et al., Biomaterials 17, 147-54 (1996)). The second approach attempts to induce repair by recruiting mesenchymal stem cells from the surrounding connective tissue, e.g., synovium, using chemotactic and/or mitogenic factors. This second approach is disclosed in, e.g., U.S. Pat. Nos. 5,206,023 and 5,270,300, each of which are herein incorporated by reference.

Chondrocytes for use in the first method are typically obtained from a low-loaded area of joint and grown in culture (see Grande; Brittberg; Shortkroff, supra), or from mesenchymal stem cells, e.g., harvested from the iliac crest marrow, and induced to differentiate along the chondrocyte lineage using growth factors (Harade et al., Bone 9, 177-83 (1988); Wakitani et al., J. Bone Joint Surg., 76-A, 579-92 (1994)). Current clinical attempts at chondrocyte transplantation are hampered because (1) they are very technically challenging, (2) the cell preparation is very expensive and (3) the potential patient pool is limited by age, defect location, history of disease, etc.

Cells have also been transplanted into cartilage defects in the form of perichondral grafts, e.g., obtained from costal cartilage. These transplantations have had limited success due to limited sources of donor material and endochondral ossification of the graft site observed in longterm follow-up (Amiel et al., Connect tissue res. 18, 27-39 (1988); O'Driscoll et al., J. Bone Joint Surq. 70-A, 595-606 (1988); Homminga et al., Acta. Orthop. Scand. 326-29 (1989); Homminga et al., J. Bone Joint Surg. 72-B, 1003-7 (1990)).

Transplanting cells into cartilage faces the difficulty of stably anchoring the cells or other repair-inducing factors within the defect site.

Recruitment of mesenchymal stem cells, the second approach, is an attractive alternative because of the availability of growth factors and cytokines in recombinant form and the lack of complicated cell transplantation. This approach also requires stably anchoring the repair-inducing factors (tissue grafts, cells, or growth factors) within the defect site.

The availability of a matrix material that can be molded to fit the lesion and also anchor the materials seeded with chondrocytes and chondrogenic factors has been a limiting factor in the repair of articular cartilage research. Unsatisfactory results have been obtained with currently available matrix materials. (Polyglycolic acid scaffolds, Kirker-Head, Clinical Orthopaedics and Related Research, 349, 205-217 (1998); Calcium phosphate minerals, Nakahara et al., Clin. Orthop. 276, 291-98 (1992); fibrin sealants, Italy et al., Clin. Orthop. 220, 284-303 (1987); collagen gels, Wakitani et al., J. Bone Joint Surg. 71-B, 74-80 (1989)).

The present invention is a novel composite material for use as a matrix to deliver cells and/or a bioactive agent e.g.: chondrocytes and transforming growth factor. The composite material is a sponge which contains a cross-linked gel that may be embedded with cells and/or bioactive agents useful to stimulate tissue repair, regeneration or augmentation. The gel-infused sponges of the present invention can provide for sustained release of bioactive agents for the promotion of cell recruitment, transformation and growth.

### SUMMARY OF THE INVENTION

The present invention relates to a composite material which is the combination of two materials: (1) a sponge and (2) a gel. The gel-infused sponge has superior handling characteristics and mechanical properties compared to the sponge or gel alone.

Another aspect of the invention is that the composite gel-infused sponge may be used to facilitate cartilage or meniscus repair in joints or to facilitate other tissue repair, regeneration or augmentation.

Another aspect of the invention is that the gel-infused sponge can be preformed, press-fit and glued into place. Alternatively, the gel and sponge can be combined so that the gel sets up in situ. In situ formation of the gel-infused sponge allows the sponge to match the geometry of the defect and interface well with the surrounding tissue.

### BRIEF DESCRIPTION OF THE DRAWINGS

FIG. 1 is a chart of the peak stress for two different gel infiltrated sponges.

FIG. 2 is a chart comparing Helistat and Integra 2K collagen sponges with cross-linked fibrinogen: stress at 50% strain

FIG. 3 is a chart comparing Helistat and Integra 2K collagen sponges with hyaluronate solution: stress at 90% strain.

### DETAILED DESCRIPTION OF THE INVENTION

This invention teaches versatile methods of regeneration and augmentation of tissue using a gel-infused matrix. The composite material of this invention is the combination of two materials: (1) a sponge and (2) a gel. This combination of sponge and gel gives handling characteristics and mechanical properties superior to either gel or sponge alone. The invention is based on the introduction of a gel which may contain bioactive agents into a sponge, typically a collagen sponge, which can be preformed and press-fit into a tissue defect or which can be set up in situ. This invention relates to the treatment and repair of damaged tissue. The gel-infused sponges of this invention are suitable as a matrix for any tissue repair or augmentation where rapid cell infiltration, remodeling, and regeneration of the damaged tissue is desirable, and where physical/spacial integrity of the repair matrix must be maintained during the remodeling phase.

Materials and Matrix Preparation

Appropriate sponges for use in this invention are collagen sponges or other wettable, biodegradable, porous scaffolds that can be molded or cut to a desired shape and can be embibed with a gel precursor. Collagen sponges for use in this invention may be purchased or may be made by procedures found in the following references which are incorporated herein by reference. Artandi disclosed a sponge comprised of acid treated swollen collagen. (U.S. Pat. No. 3,157,524, issued Nov. 17, 1964). Collins et al. disclosed an acid-swollen collagen sponge that is crosslinked by glutaraldehyde. (Surg. Forum 27:551-553 (1976)). Examples of suitable collagen sponges include Helistat (Integra LifeSciences, Plainsboro, N.J.) and other custom collagen sponges, (Integra LifeSciences, Plainsboro, N.J.), or Gelfoam (Johnson & Johnson, The Upjohn Co., Kalamazoo, Mich.). Other sponges that may be used are those consisting of polysaccharides, e.g., a hyaluronic acid sponge (Hyaff, Fidia Advanced Biopolymers, Abano Terme, Italy). A synthetic polymer such as PLGA or carboxymethyl cellulose (CMC) may be used as well.

Gels for use in the composite material of this invention consist of (i) a protein solution such as a fibrinogen or soluble collagen or a polysaccharide solution such as hyalurionic acid or a modified hyaluronic acid solution (U.S. patent application Ser. No. 09/156,829 filed Sep. 18, 1998) and if necessary, (ii) a crosslinking agent or other agent that would initiate gel formation. Examples of protein solutions suitable for use in this invention include: (1) fibrinogen solutions of 2 to 60 mg/ml that form a fibrin clot upon addition of thrombin; and (2) collagen or serum albumin solutions that form a gel upon addition of biocompatible crosslinking agents such as di- or multi-functional polyethylene glycols having functional groups that react readily with proteins such as succinimidyl esters. Soluble collagen is collagen that has an average molecular weight of less than 400,000, preferably having a molecular weight of about 300,000. A particularly soluble collagen is Cellprime or Vitrogen (Cohesion Technologies, Palo Alto, Calif.) or Semex S (Semex Medical Co., Malvern, Pa.). In general, gel formations can be initiated thermally, chemically, photo-chemically or enzymatically. Biocompatible gel initiating agents are known in the art and are typically used for crosslinking of proteins. Protein solutions that will form a gel upon addition of a biocompatible cross-linking agent include: (1) fibrinogen solutions of 2 to 120 mg/ml, preferably between 10 to 80 mg/ml and more preferably between 20 and 60 mg/ml; (2) serum albumin solutions of 10 to 300 mg/ml, preferably between 10 and 80 mg/ml and more preferably between 20 and 65 mg/ml; (3) soluble collagen solutions of 0.5 to 15 mg/ml, preferably between 1 and 6 mg/ml, and; (4) heat denatured collagen or gelatin solutions of 2 to 350 mg/ml, preferably between 5 and 150 mg/ml.

The gel precursor (comprised of a protein solution or modified polysaccharide and, if required, a cross-linking agent or gel initiating agent) is typically added to the sponge prior to its transition to a solid gel consistency. The solution is then allowed to set up in the sponge. The gel precursor infused sponge can be set up in situ or prior to being placed into the tissue defect or desired site of tissue augmentation.

Preparation of the Sponges

Sponges based on collagen or mucopolysaccharides are commercially available. For example, a collagen sponge which is suitable for use in this invention is the Helistat (Integra LifeSciences, Plainsboro, N.J.). A sponge produced from hyaluronic acid (Hyaff, Fidia Advanced Biopolymers, Abano Term, Italy) is also suitable for use in this invention.

Methods for producing collagen sponges are known in the art (U.S. Pat. Nos. 4,193,813 (Chvapil), 4,320,201 (Berg et al.) and 4,970,298 (Silver et al.)). Examples are chemical cross-linking of the collagen using a carbodiimide and cross-linking via dehydrothermal treatment. Dehydrothermal treatment is known to make the sponge stiffer and stronger. These processes, as well as cross-linking the collagen sponge with a succinimidyl active ester, and lyophilization, are described in U.S. No. Pat. No. 4,703,108 (Silver et al.). A method for forming a lyophilized biopolymer foam, into which collagen is lyophilized, giving a collagen-coated biopolymer foam is known in the art (Bell et al. U.S. Pat. No. 5,948,429).

Methods for producing polysaccharide sponges are described by Haynes et al. (U.S. Pat. No. 5,888,987), which does not involve lyophilization. Other methods are described by Dorigatti et al. (U.S. Pat. No. 5,658,582).

The density of the sponge must be low enough to allow infiltration of cells and matrix remodeling. The sponge also must be able to absorb the gel forming solution or the gel precursor readily.

General Examples of Protein Solution Prep.

Protein or other gel precursor solutions are prepared in physiological biocompatible buffer solutions as known in the art (Hubbell et al., U.S. Pat. No. 5,843,743, Barrows et al., U.S. Pat. No. 5,856,367, Rhee et al., U.S. Pat. Nos. 5,550,187, 5,523,955, 5,304,595 and Daniels et al., U.S. Pat. No. 3,949,073).

Preparation of the Gels and Addition of Bioactive Agents

A growth factor or other bioactive agent or a combination of such can be added to the sponge together with the gel precursor (e.g., the protein or polysaccharide solution, see Examples). Alternatively, the bioactive agent can be added to the sponge first, e.g., such that a bioactive agent solution is absorbed by the sponge and that subsequently, the sponge with bioactive agent are lyophilized.

Bioactive agents can be added free or encapsulated in nanospheres, PLGA microspheres, liposomes, or by other methods for the purpose of slowing down their release or of protecting them from unwanted modifications during gel setting.

Growth factors or other active agents can be added free or encapsulated in nanospheres, PLGA microspheres, liposomes or by other methods for the purpose of slowing down their release, stabilizing bioactivity or of protecting them from unwanted modifications during gel setting. Hunziker et al. encapsulated TGF-β and other growth factors into liposomes for cartilage repair (Hunziker U.S. Pat. No. 5,270,300 and Hunziker U.S. Pat. No. 5,853,746). Other investigators used sustained delivery systems for epidermal growth factor and basic fibroblast growth factor (J. Murray et al., In vitro, 19, pp. 743-748 (1983) and E. R. Edelman et al., Biomaterials, 12, pp. 619-626 (1991)). Bentz et al. (H. Bentz et al. J. Biomed. Mater. Res., 39, pp. 539-548 (1998)) reported a method of covalent binding of growth factors to a collagen matrix for the sustained release and improved stability of growth factors like TGF-β's and BMP's. Schroeder et al. and Sakiyama et al. (J. A. Schroder, H. Bentz, T. D. Estridge, J. of Controlled Release, Vol. 49, 291-298 (1997), J. A. Schroder, U.S. Pat. No. 5,693,341 and S. E. Sakiyama, J. C. Schense and J. A. Hubbell, FASEB J., 13, pp. 2214-2224 (1999)) make use of the affinity binding of growth factors like TGF-β and FGF to heparin.

Even if bioactive agents are added in free form, the density and properties of the gel within the sponge (interactions of bioactive agent with the gel or sponge such as affinity binding) can alter the kinetics of their release (Biodegradable Hydrogels for Drug Delivery, Park, Shalabay, and Park, Technomic Publishing, 1993).

The gel itself has preferably a low enough density (protein or polysaccharide concentration), so that it is readily infiltrated by cells and remodeled into new host tissue. For example for serum albumin, cell infiltration and remodeling is best at or below 80 mg/ml, and for a fibrin clot at or below 30 mg/ml, although higher concentrations may also be used.

Examples of Bioactive Agents Useful for Cartilaqe Repair

In the gel-infused sponges of this invention used for cartilage repair, one or more proliferation or mitogenic agents, chemotactic agents and/or transforming factors may be added to the gel. Proliferation or mitogenic agents stimulate the proliferation of cartilage repair cells. Chemotactic agents attract cartilage repair cells and transforming factors promote differentiation of cartilage repair cells into chondrocytes.

A proliferation or mitogenic agent is a compound or composition, including peptides, proteins, and glycoproteins, which is capable of stimulating proliferation of cells in vitro. In vitro assays to determine the proliferation (mitogenic) activity of peptides and other compounds are known in the art (see, e.g., Canalis et al., J. Clin. Invest., pp. 1572-77 (1988); Gimenez-Gallego et al., Biochem. Biophys. Res. Commun., 135, pp. 541-548 (1986); Rizzino, “Soft Agar Growth Assays for Transforming Growth Factors and Mitogenic Peptides”, in Methods Enzymol., 146A (New York: Academic Press, 1987), pp. 341-352; Dickson et al., “Assay of Mitogen-Induced Effects on Cellular Incorporation of Precursors for Scavengers, de Novo, and Net DNA Synthesis”, in Methods Enzymol., 146A (New York: Academic Press, 1987), pp. 329-340). A method used to determine the proliferation (mitogenic) activity of a compound or composition is to assay it in vitro for its ability to induce anchorage-independent growth of nontransformed cells in soft agar (e.g., Rizzino, 1987, supra). Other mitogenic activity assay systems are also known (e.g., Gimenez-Gallego et al., 1986, supra; Canalis et al., 1988, supra; Dickson et al., 1987, supra). Mitogenic effects of agents are frequently very concentration-dependent, and their effects can be reversed at lower or higher concentrations than the optimal concentration range for mitogenic effectiveness.

The proliferation agent or agents should be present in an appropriate concentration range to have a proliferative effect on cartilage repair cells in the gel-infused sponge filling the defect. Preferably, the same agent should also have chemotactic effect on the cells (as in the case of TGF-β); however, a factor having exclusively a proliferative effect may be used. In the alternative two different agents may be used, one for chemotactic cell immigration, and another for induction of cell proliferation.

Proliferation (mitogenic) agents that are useful in this invention for stimulating the proliferation of cartilage repair cells are compounds or compositions which are capable of stimulating the proliferation of cells as demonstrated by an in vitro assay, as noted above.

Particular proliferation agents that are useful include transforming growth factors (“TGFs”) such as TGF-α and TGF-β; insulin-like growth factor (“IGF I”); acidic or basic fibroblast growth factors (“FGFs”); platelet-derived growth factor (“PDGF”); epidermal growth factor (“EGF”); and hemopoietic growth factors, such as interleukin 3 (“IL-3”) (Rizzino, 1987, supra; Canalis et al., supra, 1988; Growth Factors in Biology and Medicine, Ciba Foundation Symposium 116 (New York: John Wiley & Sons, 1985); Baserga, R., ed., Cell Growth and Division (Oxford: IRL Press, 1985); Spron, M. A. and Roberts A. B. eds., Peptide Growth Factors and their Receptors, Vols. I and II (Berlin: Springer-Verlag, 1990)). However, these particular examples are not limiting.

Chemotactic agent refers to any compound or composition, including peptides, proteins, glycoproteins and glycosaminoglycan chains, which is capable of attracting cells in standard in vitro chemotactic assays (e.g. Wahl et al., Proc. Natl. Acad. Sci. U.S.A., 84, pp. 5788-92 (1987); Postlewaite et al., J. Exp. Med., 165, pp. 251-256 (1987); Moore et al., Int. J. Tiss. Reac., XI. pp. 301-307 (1989)).

Chemotactic agents useful in the compositions and methods of this invention for attracting cartilage repair cells to the cartilage defect include, for example, TGF-β, FGFs (acidic or basic), PDGF, tumor necrosis factors (e.g., TNF-α, TNF-β) and proteoglycan degradation products, such as glycosaminoglycan chains (Roberts et al. (1990), supra; Growth Factors in Biology and Medicine, Ciba Foundation Symposium 116 (New York: John Wiley & Sons, 1985); Baserga, R., ed., Cell Growth and Division (Oxford: IRL Press, 1985)).

A transforming factor or factors may also be present in the gel-infused sponge used in cartilage repair so that after cartilage repair cells have populated the porous biodegradable matrix material the transforming factor will be released into the defect site in a concentration sufficient to promote differentiation (i.e., transformation) of the cartilage repair cells into chondrocytes which form new stable cartilage tissue. Proper timing of the release of the transforming factor is particularly important if the transforming factor can inhibit or interfere with the effectiveness of the proliferation agent (see Roberts et al. (1990) supra).

Transforming factors useful in the compositions and methods of this invention to promote cartilage repair include any peptide, polypeptide, protein or other compound or composition which induces differentiation of cartilage repair cells into chondrocytes which produce cartilage-specific proteoglycans and type II collagen. The ability of a compound or composition to induce or stimulate production of cartilage-specific proteoglycans and type II collagen in cells can be determined using assays known in the art (e.g., Seyedin et al., 1985, supra; Seyedin et al., 1987, supra). The transforming factors useful in the compositions and methods of this invention include, for example, BMP's, TGF-β's, TGF-α and FGFs (acidic or basic). These transforming factors may be used singly or in combination, In addition, TGF-β may be used in combination with EGF.

Examples of Bioactive Agents Useful for Bone Repair

In the compositions of this invention used in bone repair, one or more angiogenic factors and/or osteogenic factors may be added as bioactive agents. The angiogenic factor is added to the gel infused sponge to stimulate the formation and ingrowth of blood vessels and associated cells (e.g. endothelial, perivascular, mesenchymal and smooth muscle cells) and of basement membranes in the area of the bone defect. The osteogenic factor is added to promote the growth of osteoblasts and osteocytes.

Angiogenic factor, as used herein, refers to any peptide, polypeptide, protein or any other compound or composition that induces or stimulates the formation of blood vessels and associated cells (such as endothelial, perivascular, mesenchymal and smooth muscle cells) and blood vessel-associated basement membranes. In vivo and in vitro assays for angiogenic factors are known in the art. (e.g., Gimbrone, M. A., et al., J. Natl. Cancer Inst., 52, pp. 413-419 (1974); Klangbrun, M. et al., Cancer Res., 36, pp. 110-113 (1976); Gross et al., Proc. Natl. Acad. Sci. U.S.A., 76, pp. 5217-5221 (979); Zetter. B. R., Nature (London), 285, pp. 41-43 (1980); Azizkhan R. G. et al., J. Exp. Med., 152, pp. 931-944 (1980)).

Angiogenic factors useful in the compositions and methods of this invention for stimulating vascularization throughout the gel-infused sponge in the area of the bone defect include bFGF, TGF-β, PDGF, TNF-α, angiogenin or angiotropin. Heparin sulfate has been found to enhance the angiogenic activity of bFGF (Hunziker U.S. Pat. No. 5,270,300).

An osteogenic factor may also be present in the gel-infused sponge of this invention used in bone repair in a concentration sufficient to promote a process leading to the eventual development of osteoblasts and osteocytes. The osteogenic factor may be sequestered or packaged in an appropriate delivery system within the gel-infused sponge so that it is released as the gel-infused sponge is degraded after blood vessels and associated cells have populated the gel-infused sponge.

Osteogenic factors useful in the bone repair compositions include any peptide, polypeptide, protein or any other compound or composition which induces differentiation of bone repair cells into bone cells, such as osteoblasts and osteocytes, which produce bone tissue.

The osteogenic factor induces differentiation of bone repair cells into bone cells, such as osteoblasts or osteocytes. This process may be reached through an intermediate state of cartilage tissue. The bone tissue formed from bone cells will contain bone specific substances such as type I collagen fibrils, hydroxyapatite mineral and various glycoproteins and small amounts of bone proteoglycans. Typically such osteogenic factors induce ectopic bone formation when implanted subcutaneously with demineralized bone powder (Sampath, T. K. and Reddi, A. H., Proc. Natl. Acad. Sci. U.S.A., 80, pp. 6591-6595 (1983)) or other suitable carrier materials such as collagen/ceramic composites (Bentz et al., J. Biol. Chem., 264, pp. 20805-20810 (1989)). Injection of TGF-β's subperiosteally in rats induce osteogenesis at the injection site (Noda, M. et al., Endocrinology, 124, pp. 2991-2994 (1989) and Joyce, M. E. et al., J. Cell Biology, 110, 2195-2207 (1990)).

The osteogenic factors useful in this invention include proteins such as transforming growth factor-β (Joyce, M. E. et al., J. Cell Bio., 110, 2195-2207 (1990)), osteogenin (Sampath, T. R. et al., J. Biol. Chem., 65(20), pp. 13198-13205 (1990)), (Luyten, F. P. et al., J. Biol. Chem., 264(15), pp. 13377-80 (1989)), bone morphogenetic protein (BMP) (Wang, E. et al., Proc. Natl. Acad. Sci. U.S.A., 87, pp. 2220-24 (1990)), TGF-β combined with epidermal growth factor (EGF), and other growth and differentiation factors GDF's that belong to the transforming growth factor family (Wozney et al., Clinical Orthopaedics and Related Research, 346, 26-37 (1998)).

Other bioactive agents have been found to be useful for other applications. See, e.g. Hunziker U.S. Pat. No. 5,853,746 see also, Hunziker U.S. Pat. No. 5,270,300.

The bioactive agents, either free or in a delivery vehicle, can be added first to the sponge itself, and then the bioactive agents loaded sponge is dried or lyophilized. More typically, the bioactive agents are mixed with the gel precursor prior to infusing the combination into the sponge (as described in the Examples below).

For promotion of tissue growth, regeneration or augmentation (e.g. for cartilage or bone repair) repair cells (e.g., chondrocytes or bone repair cells) may be used in the gel infused sponge instead of or in addition to bioactive agents.

Numerous investigators have seeded cells onto sponges and scaffolds made of several materials including collagen type I, collagen type II, collagen type I—glycosaminoglycan copolymer, PGA, PLA, PLA-PGA copolymers, nylon, carbon fiber and alginate (Nehrer et al., Biomaterials, 18, pp. 769-776 (1997); Doillon et al., Biomaterials, 8, pp. 195-200 (1987); Toolan et al., Journal of Biomed. Mat. Res., 31, pp. 273-280 (1996); Chu et al., Journal of Biomed. Mat. Res., 29, pp. 1147-1154 (1995); Grande et al., Journal of Biomed. Mat. Res., 34, pp. 211-220 (1997); Fujisato et al., Biomaterials, 17, pp. 155-162 (1996); Shapiro et al., Biomaterials, 18, pp. 583-590 (1997)). The effects of sponge or scaffold composition and structure on cell behaviors such as attachment, alignment, viability, proliferation, and biosynthesis were evaluated in culture studies. In two of these studies, materials were implanted into animals after in vitro culture of cell seeded constructs (Chu et al., Journal of Biomed. Mat. Res., 29, pp. 1147-1154, (1995); Fujisato et al., Biomaterials, 17, pp. 155-162 (1996)). In both of these cases the cells appeared to maintain or develop a chondrocytic phenotype.

Combining and Crosslinking Procedure

As described in Examples 1 and 2 below, in general, when a gel initiating agent such as thrombin, tissue transglutaminase or a di- or multifunctional PEG succinimidyl ester is necessary, it is added to the protein or modified polysaccharide solution immediately prior to infusion into the sponge. The gel-infused sponge is then left to set up before implantation, or more preferably is set up in situ. The set up time is typically between 2-10 minutes, but can be changed depending on the gel density and the concentration of the gel initiating agent. Gel initiation can be triggered enzymatically, e.g., by thrombin or tissue transglutaminase, thermally, photo-chemically, or chemically, e.g., by di- or multifunctional succinimidyl esters.

Preparation of Gel-Infused Sponges

Typically the gel precursor containing growth factors or other bioactive agents is added into the sponge immediately following the initiation of gel formation (e.g., addition of a crosslinking agent or thrombin), when such an agent is used, and is allowed to set up within the sponge. Growth factors or other bioactive agents are added free or in a suspension encapsulated in liposomes, nanospheres or microspheres. The gel-infused sponge can be allowed to set up prior to being placed into the cartilage defect and can then be press-fit and glued into place. More preferably, the gel-infused sponge can be allowed to set up in situ. For subcutaneous evaluation in the rat model (see example B), all the gel-infused sponges were allowed to set up prior to implantation.

### EXAMPLES

Materials and Abbreviations:

Helistat: Helistat collagen sponge, Integra LifeSciences, Plainsboro, N.J.

Integra.2K: Collagen sponge with increased collagen concentration as compared to the Helistat, Integra LifeSciences, Plainsboro, N.J.

HA: Hyaluronic acid, sodium salt, MW 1-1.8 million, Genzyme Pharmaceuticals, Boston

FB: bovine fibrinogen, Sigma Chemical, St. Louis

BSA: bovine serum albumin, Sigma Chemical, St. Louis

CIS: bovine collagen-in-solution, Cohesion, Palo Alto

SC4PEG: (SC)4-PEG 20 kD, Shearwater Polymers

SPAPEG: (SPA)2-PEG 3.4 kD, Shearwater Polymers

All numbers in front of the abbreviations FB, BSA, CIS, HA, HAED or SC4PEG and SPAPEG indicate the final concentration in mg/ml of this component in the formulation.

Preparation of Gel-infiltrated Sponges

### Example 1

### Fibrin Clot Reinforced with a Collagen Sponge: Helistat-10FB-Thrombin

A piece of Helistat collagen sponge was cut to the same size or slightly larger than the defect size it was implanted in for cartilage or bone defect model (for example, goat articular cartilage defect). To a physiological buffer solution of 5-30 mg/ml fibrinogen which contains growth factor, e.g.: rhBMP-2 at 250-300 μg/ml, a small amount of a 50 U/ml thrombin solution was added (e.g., 20 μl thrombin solution per 300 μl fibrinogen solution). The sponge was then immersed in the fibrinogen/growth factor/thrombin solution prior to clotting. The gel precursor filled sponge was gently pressed to extrude any entrapped air and was allowed to set up prior to subcutaneous implantation into rats (see below, example A).

### Example 2

### Cross-Linked Fibrinogen Gel Reinforced with a Collagen Sponge: Helistat-30FB-4SC4PEG

In this example, 228 μl of 75% glycerol in water-for-injection (WFI) was added to a solution of 600 μl of 50 mg/ml fibrinogen in 50 mM sodium phosphate and 100 mM sodium chloride, pH 7.5. Added to this solution was 60 μl of rhBMP-2 at 4.4 mg/ml in 0.01 N hydrochloric acid (HCl), pH 2.0. In addition, rhTGF-β (e.g.: 12 μl, of 0.5 mg/ml TGF-β in 0.01 N HCl/35% ethanol) was added. Just prior to implantation, 100 μl of freshly dissolved SC4PEG in WFI was added to the fibrinogen/growth factor solution. Then, the gel precursor, namely the fibrinogen solution containing growth factors and the cross-linker, was immediately added to a pre-cut collagen sponge (in the same manner as Example 1) so that the sponge was maximally swollen. For subcutaneous implantation into rats 150-170 μl of gel precursor was added to a 8 mm diameter, 2-3 mm thick Helistat or Intregra 2K collagen sponge, and allowed to set up (about 5 minutes) prior to implantation. For implantation into an osteochondral defect in goats (see example B), the gel-precursor imbibed sponge was allowed to set up in situ.

### Example 3

### Collagen Gel Reinforced with a Collagen Sponge: Helistat-1.3CIS-12SC4PEG

In this example, 21 μl of 4.3 mg/ml rhBMP-2 in 0.01 N HCl and 4 μl of 0.5 mg/ml rhTGF-β2 in 0.01 N HCl/35% ethanol was added to a solution of 125 μl of 3 mg/ml soluble collagen in 0.01 N HCl (Vitrogen, Cohesion, Palo Alto, Calif.). Next, 110 μl of 0.2 M phosphate in 40% glycerol/WFI, at pH 7.6 was added. Then 80 μl of 50-100 mg/ml SC4PEG in WFI was added.

The gel precursor, i.e., the collagen/growth factors/cross-linker solution, was immediately added to a pre-cut sponge (see Examples 1 and 2) so that the sponge was maximally swollen and allowed to set up for approximately 8-10 minutes prior to further manipulation in the same manner as examples 1 and 2.

### Example 4

### Serum Albumin Gel Reinforced with a Collagen sponge:

a) Helistat-235BSA-36SPAPEG

b) Helistat-60BSA-34SC4PEG

a) The Helistat-235BSA-36SPAPEG was prepared by adding 26 μl of rhBMP-2 stock solution (4.3 mg/ml in 0.01 N hydrochloric acid) to 326 μl of 300 mg/ml serum albumin (BSA, Sigma, St. Louis) in phosphase buffered saline (PBS). Prior to implantation, 65 μl of 230 mg/ml SPAPEG in WFI was added. Then, the gel precursor was added immediately to the precut sponge as described in the previous examples, and allowed to set up for 3-6 minutes prior to further manipulation in the same manner as examples 1 and 2.

b) The Helistat-60BSA-34SC4PEG was prepared by adding 24 μl of rhBMP-2 stock solution (4.3 mg/ml in 0.01 N hydrochloric acid) of 212 μl of 100 mg/ml serum albumin (BSA) in phosphate buffered saline (PBS). Prior to implantation, 120 μl of 100 mg/ml (SC4PEG in WFI was added. Then, the gel precursor was immediately added to the precut sponge as described in the previous examples, and allowed to set up for 3-6 minutes prior to further manipulation in the same manner as examples 1 and 2.

### Example 5

### Non-Cross-Linked Hyaluronic Acid (HA) Reinforced with a Collagen Sponge: Helistat-3HA in Glycerol/Phosphate Buffer

This procedure utilizes a viscous solution of sodium hyaluronate in a glycerol containing phosphate buffer, and therefore does not require a cross-linker or gel-initiating agent (see Table 1, numbers 2 and 4).

Sodium hyaluronate (Genzyme Pharmaceuticals) was dissolved at 1.5-4 mg/ml in a solution of 0.05-0.1 M phosphate buffer at pH 7.4 containing 15-40% glycerol. Growth factors were added as described in examples above. The viscous liquid was added onto the precut sponge so that the sponge was maximally swollen. This takes approximately 1-5 minutes depending on the viscosity of the hyaluronic acid solution and the size of the Helistat sponge.

### Example 6

### Modified Hyaluronic Acid Gel Reinforced with a Collagen Sponge: Helistat-2.6HAED-2SC4PEG

In this example 20 μl of 10.4 mg/ml rhBMP-2 in 0.01 N HCl and 4 μl of 1 mg/ml rhTGF-β2 in 0.01 N HCl/35% ethanol was added to 260 μl of a 8 mg/ml solution of ethylene diamine modified hyaluronic acid in WFI (Aeschlimann et al., U.S. patent application Ser. No. 09/156,829 filed Sep. 18, 1999). Then 60 μl of WFI and 340 μl of 0.1 M phosphate, pH 7.2, containing 40% glycerol was added. Just prior to implantation, 115 μl of 14 mg/ml SC4PEG freshly dissolved in WFI was added, so that the final concentration of SC4PEG in the precursor gel was 2 mg/ml.

Immediately after addition of the SC4PEG, the gel precursor was added to the sponge, and allowed to set up for about 2-3 minutes prior to further manipulation in the same manner as examples 1 and 2.

Testing of Gel-Infiltrated Sponges

### Example A

### Measurement of Mechanical Strength

Appropriate mechanical function of repair tissue is the critical goal in, e.g., articular cartilage repair. The physical properties of articular cartilage in vitro have been extensively studied (Mow et al., J. Biomech. Eng., 102, pp. 73-84 (1980); Lee et al., J. Biomech. Eng., 103, pp. 280-292 (1981); Kempson et al., Biochem. Biophys. Acta., 215, pp. 70-77 (1970), Frank et al., J. Biomechanics, 20, pp. 629-639 (1987)). Many investigations have also assessed “in situ” physical properties of articular cartilage using portable indenter probes (Dashefsky, Arthroscopy, 3, pp. 80-85 (1987); Athanasiou et al. U.S. Pat. No. 5,503,162; Kiviranta et al. U.S. Pat. No. 5,494,045). Thus the mechanical characteristics of normal as well as pathologic articular cartilage are documented.

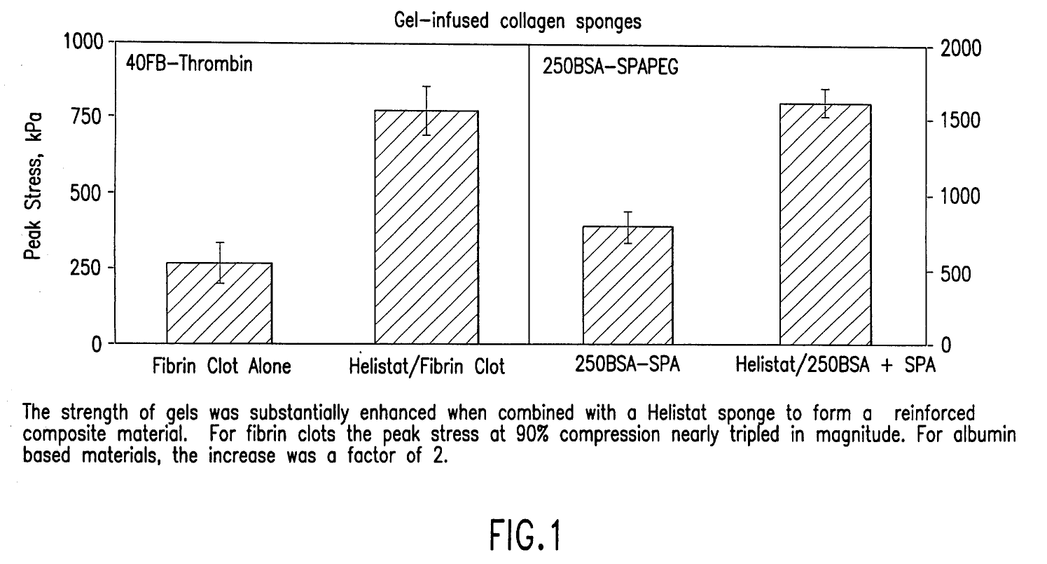

However, the mechanical requirements of an implant material for cartilage repair are not well defined. It is not clear that a scaffold material suitable for cartilage repair or tissue regeneration must initially possess identical mechanical properties as the desired tissue (cartilage) itself. In fact, it would seem that the characteristics that give rise to the desired mechanical properties might indeed interfere with adequate repair (e.g. high density, highly cross-linked gel materials are quite strong but appear to inhibit cell infiltration and subsequent repair. See FIG. 1 and section on rat subcutaneous assay).

The minimum requirement is thus for a mechanically stable material that can withstand surgical handling and implantation and will maintain its volume and shape once implanted until subsequent resorption and replacement by repair tissue. As a first order evaluation, we have made a simple axially confined, radially unconfined compressive measurement of strength. By doing so, we are including effects from all components of the materials: those acting to resist tension (radially), as well as those that resist compression (axial) (Armstrong et al., J. Biomech. Eng., 106, pp. 165-173 (1984); Kim et al., Arch. Biochem. Biophys., 311(1), pp. 1-12, (1994). This will provide information about relevant failure modes for each material, in particular when used to repair large surface area shallow lesions in which the mechanical challenge to an implant material is likely to be greater than for small diameter deep lesions.

Initial Qualitative Evaluation

Sponges were punched into 0.8 cm diameter cylindrical discs with thicknesses ranging from 2 to 4.5 mm, and soaked with various protein solutions to form a “gel within the sponge” (or gel-infused sponge). Visual observations were made of the time required for gels to form within the sponges, and qualitative general physical handling characteristics like firmness and the ability to retain the original shape upon handling and pressing were noted.

Mechanical Measurement Testing Method (Compression Test)

The matrix materials (sponges, gels and gel-infused sponges) used for mechanical evaluation were cylindrical disks of 4, 6 or 8 mm diameter. Mechanical evaluation of matrix materials was performed using a Texture Analyzer System (Texture Technologies, Scarsdale, New York) equipped with a 5 kg load cell and Texture Expert v1.12 software. Radially unconstrained specimens were placed on the test platform of the instrument and the 25 mm diameter perspex loading platen was lowered to make contact with the top of the test specimen (a trigger force of at least 1 g was used). Sample height was noted. The platen was then lowered at a rate of 100 μm/sec to the desired distance of travel (level of compression) and subsequently elevated at a rate of 3 mm/sec. Materials were generally tested to 90% compression. The stress required to initiate failure (strength) was obtained along with the strain (% compression) at failure. The threshold value for failure was a force drop of 10 grams. For cases in which the test specimen did not fracture, the peak stress at 90%, and the strain were used for comparison to other materials. For most materials, the number of samples, n, ranged from 4 to 6.

Mechanical Strength Testing Results

The results of the mechanical measurements are reported in Table 1. See FIGS. 1, 2, and 3; the standard deviations are relatively low and differences in strength are significant.

TABLE 1
Comparison of the compressive strength of a) collagen sponges
wetted with PBS or HA, b) gels formed within the sponge
and c) protein gels alone

| # | Material | Strain % | Stress kPa |
|---|---|---|---|
| 1 | Helistat-PBS | 89.1 | 3.8 |
| 2 | Helistat-3.3 HA in PBS/glycerol | 90.8 | 47.2 |
| 3 | Integra 2K-PBS | 90.5 | 64.0 |
| 4 | Integra 2K-3.3 hyaluronate in PBS/glycerol | 89.1 | 201.9 |
| 5 | Fibrin Clot (40 mg/ml) | 87.5 | 268.5 |
| 6 | Helistat-Fibrin Clot (40 mg/ml) | 89.2 | 775.6 |
| 7 | 250BSA-36SPA-PEG | 89.5 | 789.1 |
| 8 | Helistat-250BSA-36SPA-PEG | 83.4 | 1615.3 |
| 9 | 36FB-5.4 SC4-PEG | 91.3 | 179.46 |
| 10 | Helistat-36FB-5.4 SC4-PEG | 58.5 | 246.32 |

Compression measurements were performed on the gels either alone or as part of the gel-Helistat sponge composite (Table 1, numbers 5-10). The gels evaluated were fibrin clots, cross-linked fibrinogen, or cross-linked bovine serum albumin. The strength of the fibrin clot and cross-linked albumin gels was substantially increased when the gels were incorporated into the Helistat sponge to form a reinforced composite material (Table 1, numbers 5-8 and FIG. 1).

The peak stress at ˜90% compressive strain was measured for Helistat or Integra 2K collagen sponges soaked with phosphate buffered saline (PBS) or sodium hyaluronate (HA) in a phosphate buffered solution containing glycerol. Adding the viscous hyaluronate solution as described in Example 5, above, led to a substantial increase in peak stress at 90% compression compared to the sponges wetted with PBS alone: 12 fold greater for the Helistat sponge and 3 fold greater for the denser Integra 2K collagen sponge (Table 1, lines 1-4).

The peak stress of fibrin clots at 90% compression nearly tripled in magnitude when formed within the sponge scaffold. Although the albumin-based matrix alone was as strong as the reinforced fibrin clot, forming the albumin gel within the Helistat sponge still resulted in a two-fold increase of strength. Reinforcement of the cross-linked fibrinogen gel by incorporating it into a Helistat sponge lead to a 40% increase in strength (Table 1, numbers 9-10).

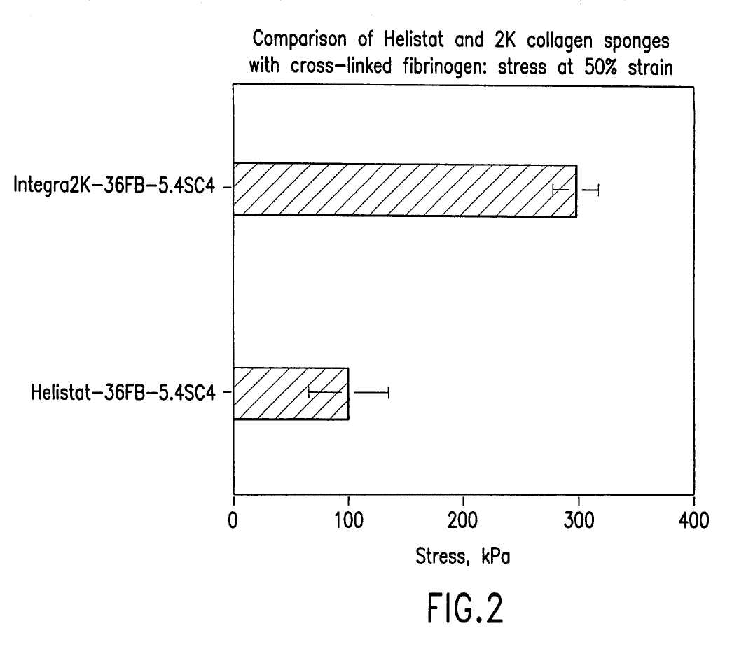

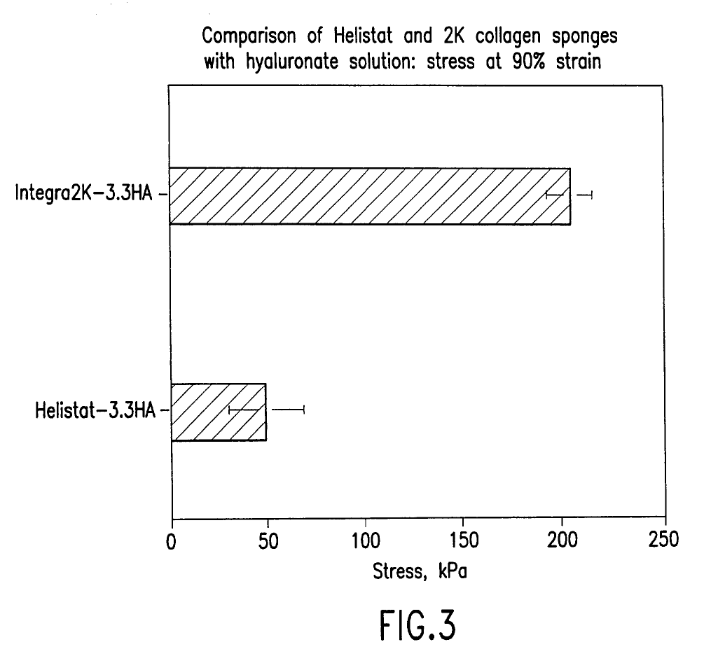

Mechanical properties of the gel-sponge composite can also be altered by selecting a sponge with specific characteristics. These characteristics can include stiffness as well as strength. As indicated in Table 1, line numbers 1 and 3, the wetted Integra 2K sponge is stiffer than the wetted Helistat sponge. The measured peak stress of cross-linked fibrinogen within each of the sponge samples at 50% compression was almost 3-fold higher for samples reinforced with the Integra 2K sponge as compared to the Helistat sponge (FIG. 2). When we compared the two sponges with a hyaluronate solution, the increase in peak stress at 90% strain was four times higher for the Integra 2K-HA combination as compared to the Helistat-HA combination (FIG. 3). These results indicate that one can specify requirements for a material and optimize each component, either the gel or the sponge, to attain the desired mechanical properties of the composite.

### Example B

### In vivo Assay for Growth Factor-Induced Ectopic Bone Formation

Procedures:

Gel-infused sponges were screened in vivo using the rat ectopic bone formation model (Reddi, J Rheumatol Suppl, 11, 67-69 (1983), Wang et al., Proc. Natl. Acad. Sci. USA, 87, 2220-2224 (1990), Wozney et al., Science, 242, 1528-34 (1998)). This assay primarily evaluates the ability of materials to remodel into bone, when combined with bone morphogenetic protein (BMP) which is reflected in the bone scores given in Table 2. This assay also assesses adverse reactions to the materials such as excessive inflammation or fibrosis that would counteract bone induction. The amount of residual matrix gives an assessment of matrix resorption rates relative to bone formation rates. Since in this model, BMP activity is enhanced by TGF-β, both BMP-2 and TGF-β2, were added into the matrix (Bentz et al., Matrix, 11, 269-275 (1991)).

Three to four week old male Sprague-Dawley rats were anaesthetized and the ventral abdomen and thorax were shaved and wiped with alcohol. A small skin incision was made on either side of the xyphoid process. The skin was separated from the subcutaneous tissue using hemostatic foreceps for about 2-3 cm, so that a pocket was created over the ventral thorax.

For this assay, sponge discs (0.8 cm diameter; 2-3.5 mm thickness) were cut and subsequently imbibed with a gel precursor as set forth in Table 2. The sponge gel composites were then allowed to fully set up prior to implantation. The implant material was placed at the cranial end of each undermined pocket. The incision was closed with one staple.

At least two to three animals were implanted with each material and each animal received two implants of the same material. The implant materials were explanted 10-14 days after implantation. Gross observations were noted and explants were fixed in 10% neutral buffered formalin, decalcified as necessary, and embedded in paraffin.

Semiserial sections through the center of retrieved implants were stained with Gomori's Trichrome or H+E. Slides were evaluated microscopically by a board certified pathologist, and scored for bone formation, fibrosis and presence of residual matrix. The amount/quality of bone was scored from 0-3 with 3 representing the most abundant bone. Similarly for fibrosis, a score of 3 represents the most abundant fibrosis. All materials contained rhBMP-2, 220-300 μg/ml, and rhTGF-β2, 6-8 μg/ml, to stimulate cellular infiltration into the implant and remodeling of the implant into cartilage and bone. In this model bone forms via the endochondral bone formation pathway (therefore some cartilage may be observed at this early time point).

Results and Conclusions

The degree of bone formation, presence of cartilage/fibrocartilage, extent of fibrosis and amount of residual matrix of gel-infused sponges were compared to a control sponge (Table 2, number 1). The control Helistat collagen sponge was prepared by first soaking the sponge in a rhBMP-2/rhTGF-β2 solution. Then the sponge was freeze-dried and implanted. Results from histologic evaluations are summarized in Table 2.

TABLE 2
Histologic evaluation of growth factor induced ectopic bone formation of
gel-infused collagen sponges in the rat subcutaneous implantation model

| | | | Cartilage | | |
|---|---|---|---|---|---|
| | | | (C) | | |
| | | | Fibrocart. | Fi- | Matrix |
| # | Matrix Materials | Bone | (FC) | brosis | present |
| 1 | Helistat-lyophilized with | 3+ | C | 1 | little |
| | growth factors (control | | | | |
| | sponge, without a gel) | | | | |
| 2 | Helistat + 3.8 HA/glycerol | 3+ | C | 1 | trace |
| 3 | Integra 2K sponge + 2.8 | 3 | C | 2 | trace |
| | HA/glycerol | | | | |
| 4 | Helistat + 2.8 HA/glycerol | 3− | C | 2 | little |
| 5 | Helistat-40 Fibrin Clot | 2-3 | FC | 1 | P |
| 6 | Helistat-60 Fibrin Clot | 2+ | FC | 2 | P |
| 7 | Helistat-100 Fibrin Clot | 1 | FC | 3+ | P |
| 8 | Helistat-1.3 CIS-22 | 2-3 | FC/C | 1 | P |
| | SC4PEG | | | | |
| 9 | Helistat-1.4 CIS-SC4PEG | 2-3 | C | 1 | P |
| 10 | Helistat-2.2 CIS-13 | 3 | C | 2 | P |
| | SC4PEG | | | | |
| 11 | Helistat-2.2 CIS-1.2 HA-13 | 3 | C | 1 | little |
| | SC4PEG | | | | |
| 12 | Helistat-2.3 CIS-10BSA-18 | 2− | C | 2 | P |
| | SC4PEG | | | | |
| 13 | Helistat-235 BSA-36 | 0 | 0 | 2 | P |
| | SPAPEG | | | | abundant |
| 14 | Helistat-20 BSA-24 | 1+ | FC | 1 | P |
| | SC4PEG | | | | |
| 15 | Helistat-20 BSA-17 | 1-2 | FC | 1 | P |
| | SC4PEG | | | | |
| 16 | Helistat-28 BSA-1.7HA-35 | 1 | FC | 1 | P |
| | SC4PEG | | | | |
| 17 | Helistat-33 BSA-18 | 1− | FC | 1− | P |
| | SC4PEG | | | | |
| 18 | Helistat-60 BSA-35 | 1+ | C | 1+ | P |
| | SC4PEG | | | | |
| 19 | Helistat-22 FB-6 SC4PEG | 2− | FC/B | 3 | P |
| 20 | Helistat-28 FB-4.7 SC4PEG | 2 | FC | 3 | P |
| 21 | Helistat-36 FB-5.4 SC4PEC | 3− | FC/C | 3 | P |
| 22 | Helistat-50 FB-7.5 SC4PEG | 2 | FC | 2 | P |

Histological score: 0-3, 3 = most abundant bone or fibrosis

All the gel-infused sponge materials appeared to be well tolerated by normal surrounding tissues. In general, implants were surrounded by a thin fibrous capsule. Inflammation of the surrounding tissue was absent or was mild and consisted primarily of mononuclear cells.

The control Helistat sponge and the Helistat sponges soaked in a hyaluronate/glycerol solution containing growth factors (see Example 5), demonstrated good cell infiltration and the most extensive bone formation among the materials studied (Table 2, numbers 1, 2, 3 and 4) at 10 to 11 days post-implantation. This was evident because the implanted matrix remodeled almost entirely into solid bone surrounding little residual sponge matrix in the center of the implant. Osteoblasts and osteoclasts were prominent, indicating active formation and remodeling of bone. Cartilage was present at the inner margin of bone within the implant, which is the expected transition in an endochondral bone formation process. Mild mononuclear cell infiltration, capillary ingrowth and fibroblast proliferation were typically associated with the residual matrix in the center of the implant.

For Helistat sponges filled with a fibrin clot, bone formation was abundant for fibrin at 40 mg/ml concentration and decreased with increasing fibrin concentrations. Fibrocartilage and fibrosis increased with increasing fibrinogen concentrations (see Example 1 and Table 2, numbers 5, 6 and 7). At 100 mg/ml fibrin, substantially less bone was present in a predominantly fibrous implant.

Helistat sponges filled with soluble collagen (CIS) and cross-linked with the high molecular weight branched SC4PEG (see Example 3), formed a thick solid ring of bone which derived from well-developed cartilage located at the inner margin of the ring. Residual, moderately infiltrated matrix was present at the center of the implant (Table 2, numbers 8, 9 and 10). The addition of bovine serum albumin to the Helistat-CIS matrix increased gel strength but resulted in a lower degree of bone formation (Table 2, number 12).

Helistat sponge filled with bovine serum albumin (BSA) and cross-linked with either SPAPEG or the branched SC4PEG (17-36 mg/ml) resulted in little to moderate bone formation (Example 4, Table 2, numbers 13-18). No bone formed and cell infiltration was poor with the dense, high concentration BSA matrix, 200-250 mg/ml (Table 2, number 13).

Similarly, fibrinogen cross-linked with the branched SC4PEG (4.7 to 7.5 mg/ml) within the Helistat sponge, showed moderate to good bone formation with residual matrix at the center of the implant, in a concentration range between 20-50 mg/ml fibrinogen (Example 2, Table 2, lines 19-22).

Gel-infused sponges filled with gels of low to moderate density, demonstrate abundant bone and cartilage formation and minimal fibrosis. With increasing gel density or higher protein or gel precursor concentrations, bone formation was substantially lower or absent (Table 2, numbers 7, 13), as observed for high density gels without sponges.

This study demonstrates that gels of low cross-link density and/or of low protein or gel precursor concentration, that would form only weak gels by themselves: (1) form a more cohesive and stronger material when added into a sponge; and, (2) retain enough porosity to be remodeled into the new tissue, in this case, bone.

### Example C

### In vivo Evaluation of Helistat Gel-Sponge Composites for Repair of Osteochondral Lesions

Animals and Procedures:

Skeletally mature, female, Spanish goats weighing between 30-45 kg were used. Animals were divided equally into 3 groups. The three groups received the two matrices to be evaluated and the third group was the control with unfilled lesions. In each goat, two 5.5 mm diameter×8 mm deep full thickness osteochondral lesions were created in the trochlear sulcus, one proximally and the other distally. Thrombin (40 μl at 50 U/ml) was used to stop bleeding as needed.

Matrix 1 consisted of the Helistat sponge containing cross-linked fibrinogen similar to that described in Example 2 above: Helistat-30FB-4SC4PEC, 17% glycerol.

Matrix 2 consisted of the Helistat sponge containing the cross-linked soluble collagen similar to that described in Example 3 above: Helistat—1.35 CIS-12SC4PEG, 14% glycerol.

Both materials contained 250 μg/ml rhBMP-2 and 40 ng/ml rhIGF-1. For implantation, the gel-infused sponges were implanted to set up in situ.

Postoperative Treatment:

Animals were allowed to bear weight as tolerated. No immobilization or splinting was used. At 8 weeks, all animals were euthanized. The joints were grossly evaluated, photographed, and fixed in 10% neutral buffered formalin. Samples were embedded in plastic, and slides prepared by the cutting-grinding method. Slides were evaluated by a pathologist, and scored using a scale of 0-3 for 16 different criteria.

Results:

Implants were evaluated and scored. Score results are summarized in Table 3 below:

TABLE 3
Histologic evaluation of growth factor induced bone repair of gel-
infused collagen sponges in a goat osteochondral defect model.

| | | | Total Bone Score |
|---|---|---|---|
| Matrix | Time Point | n | (24 points max.) |
| Helistat-30FB-4SC4PEG | 8 weeks | 12 | 13.5 ± 1.18 |
| Helistat-1.35CIS-12SC4PEG | 8 weeks | 6 | 17.83 ± 2.76 |
| Empty | 8 weeks | 10 | 17.7 ± 1.61 |

The invention claimed is:

1. A gel-infused sponge matrix comprising a porous biodegradable sponge material infused with a gel precursor.

2. The gel-infused sponge matrix of claim 1 wherein the porous biodegradable sponge material is of a density to allow cell infiltration and remodeling for repair, regeneration or augmentation of tissues.

3. The gel-infused sponge matrix of claim 1 wherein the gel precursor has been cross-linked by a gel-initiating agent.

4. The gel-infused sponge matrix of claim 1 wherein the gel-precursor material contains bioactive agents.

5. The gel-infused sponge matrix of claim 1 wherein the porous biodegradable sponge material is comprised of collagen.

6. The gel-infused sponge matrix of claim 1 wherein the porous biodegradable sponge material is comprised of polysaccharides.

7. The gel-infused sponge matrix of claim 1 wherein the porous biodegradable sponge material is comprised of a synthetic polymer.

8. The gel-infused sponge matrix of claim 1 wherein the porous biodegradable sponge material is comprised of hyaluronic acid or modified hyaluronic acid.

9. The gel-infused sponge matrix of claim 1 wherein the gel precursor is comprised of fibrinogen and thrombin.

10. The gel-infused sponge matrix of claim 1 wherein the gel precursor is comprised of a fibrinogen solution having a concentration of about 10 to 80 mg/ml.

11. The gel-infused sponge matrix of claim 1 wherein the gel precursor is comprised of a serum albumin solution.

12. The gel-infused sponge matrix of claim 1 wherein the gel precursor is comprised of a serum albumin solution having a concentration of about 10-80 mg/ml.

13. The gel-infused sponge matrix of claim 1 wherein the gel precursor is comprised of soluble collagen.

14. The gel-infused sponge matrix of claim 1 wherein the gel precursor is comprised of a soluble collagen having a concentration of about 1-6 mg/ml.

15. The gel-infused sponge matrix of claim 1 wherein the gel precursor is comprised of heat denatured collagen.

16. The gel-infused sponge matrix of claim 1 wherein the gel precursor is comprised of a heat denatured collagen having a concentration of about 5-150 mg/ml.

17. The gel-infused sponge matrix of claim 1 wherein the gel precursor is comprised of gelatin.

18. The gel infused sponge matrix of claim 1 wherein the gel precursor is comprised of hyaluronic acid or polysaccharides.

19. The gel-infused sponge matrix of claim 1 wherein the gel precursor is comprised of hyaluronic acid or polysaccharides having a concentration of about 1-8 mg/ml.

20. The gel infused sponge matrix of claim 1 wherein the gel precursor is comprised of modified hyaluronic acid or polysaccharides.

21. The gel-infused sponge matrix of claim 1 wherein the gel precursor is comprised of modified hyaluronic acid or polysaccharides having a concentration of about 1-8 mg/ml.

22. A process for the preparation of a gel-infused matrix material, wherein said gel-infused matrix material is comprised of a porous biodegradable matrix material infused with a gel precursor, and wherein the process is comprised of the following steps:
(1) soaking said porous biodegradable matrix material in a gel containing bioactive agents;
(2) compressing said porous biodegradable matrix material to extrude out air; and
(3) slowly releasing the matrix allowing the porous biodegradable matrix material to fully absorb the gel precursor.

23. The process of claim 22 wherein the porous biodegradable matrix material is cut to the required size before soaking said porous biodegradable matrix material in a gel precursor containing bioactive agents.

24. The process of claim 22 wherein the porous biodegradable matrix material is cut to the required size after said porous biodegradable matrix material has been infused with the gel precursor.

25. The process of claim 22 for the preparation of a gel-infused matrix material wherein the process is performed in-situ.

26. The process of claim 22 for the preparation of a gel-infused matrix material wherein the process is performed in-vitro and then implanted.

27. The process of claim 22 for the preparation of a gel-infused matrix material wherein the gel initiating agent is added to the gel precursor.

28. A method of implanting a gel-infused matrix material for the promotion of tissue growth, regeneration, repair or augmentation.

29. A method of implanting a gel-infused matrix material for the promotion of bone, cartilage, meniscus or nerve growth, regeneration, repair or augmentation.

30. The gel infused sponge matrix of claim 1 wherein the gel infused sponge also contains repair cells.
